(19) United States
(12) Plant Patent Application Publication
(10) Pub. No.: US 2014/0109270 P1
(43) Pub. Date: Apr. 17, 2014

(54) BLUEBERRY PLANT NAMED "BB06-507MI-15

(50) ;

(71) Applicant: BERRY BLUE, LLC, Grand Junction, MI (US)

(72) Inventors: Edmund J. Wheeler, Holland, MI (US); James F. Hancock, East Lansing, MI (US)

(73) Assignee: BERRY BLUE, LLC, Grand Junction, MI (US)

(21) Appl. No.: 13/653,264

(22) Filed: Oct. 16, 2012

(51) Int. Cl. A01H 5/00

(52) U.S. Cl. PLT/157

(57) ABSTRACT

A new and distinct cultivar of blueberry plant named BB06-507MI-15 as described and shown herein. BB065-507MI-15 is a new and distinct high chill tetraploid Northern highbush blueberry (Vaccinium) variety of complex ancestry, based largely on V. corymbosum with a limited number of genes from V. darrowii, V. angustifolium, V. tenellum, and V. ashei. It is a productive early ripening variety that ripens approximately at the same time as Duke, and 10 days before Bluecrop. BB065-507MI-15 provides a very good yield potential, a very good plant vigor, fruit quality and firmness, as well as characteristics suitable to mechanical harvesting, and very good flavor and texture.

### BACKGROUND AND SUMMARY

Blueberries are a well-known fruit enjoyed by many throughout the world. One example of an existing, patented blueberry variety is Bluecrop, unpatented. Another example of an existing, patented blueberry variety is Elliott, also unpatented.

Compared to Bluecrop, the maturity date of the present cultivar, BB06-507MI-15, is 10 days earlier than Bluecrop, and the new cultivar has a much lighter blue color. BB06-507MI-15 is slightly larger, has a much firmer berry, and a bush habit that is much more upright than Bluecrop.

Compared to Elliott, the maturity date of BB06-507MI-15 is 6 weeks earlier. BB06-507MI-15 has a much lighter blue color and a larger, much firmer berry. The bush shape of BB06-507MI-15 is more upright than Elliott.

The present cultivar, BB06-507MI-15, provides one or more advantages compared to these and/or other blueberry varieties.

### BRIEF DESCRIPTION OF THE PHOTOGRAPHS

FIG. 1 is a close-up photograph taken in June 2012 of the Blueberry cultivar BB06-507MI-15, showing mature leaves and also showing the shape and color, petiole, and venation of leaves, and also the immature cane color.

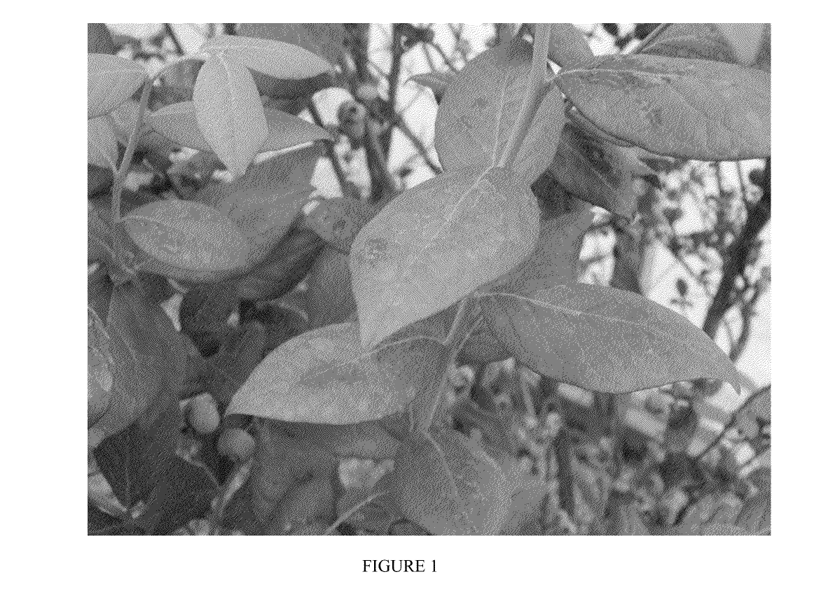

FIG. 2 is a photograph taken in June 2012 of the Blueberry cultivar BB06-507MI-15, showing a 6 year old bush in pot, and showing the bush shape, branching habit, color of leaves, and fruit presentation of the variety.

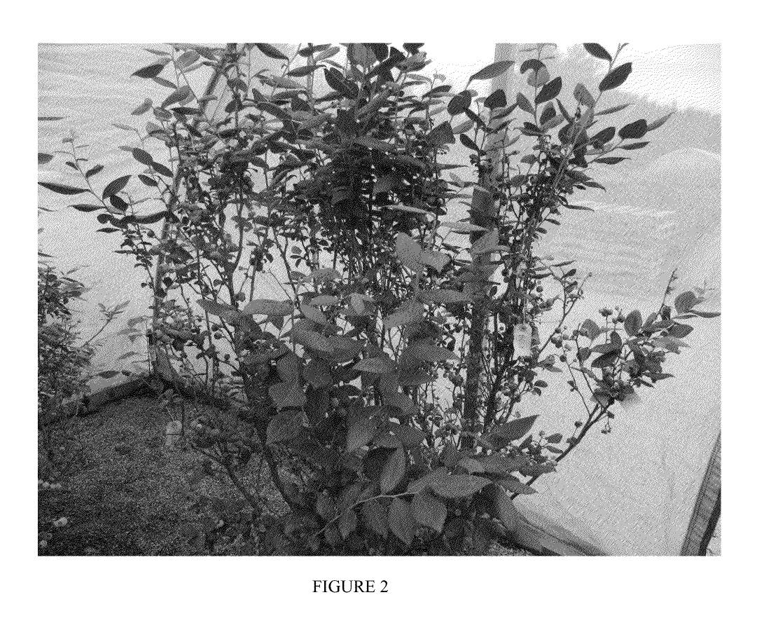

FIG. 3 is a close-up photograph taken in June 2012 of the Blueberry cultivar BB06-507MI-15, showing a fruit cluster with ripe and unripe fruit, and also showing shape, calyx, pedicel, peduncle and cluster density of the fruit.

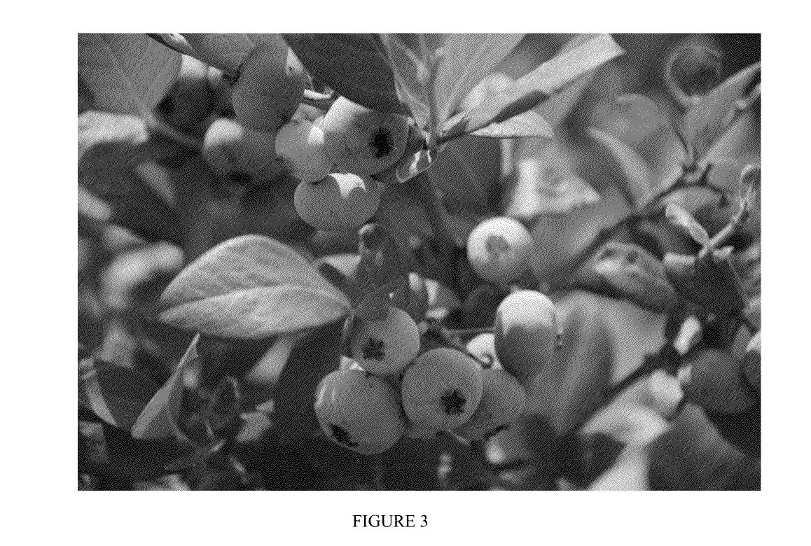

### DETAILED DESCRIPTION

Note: statements of characteristics herein represent exemplary observations of the cultivar herein and will vary depending on time of year, location, annual weather, etc. Where dimensions, sizes, colors, and other characteristics are given, it is to be understood that such characteristics are approximations and averages. The descriptions reported herein are largely from specimen plants grown near Grand Junction, Mich. Data were obtained on plants that were 3-6 years old.

Cultivar Name: BB06-507MI-15.

Classification:

### Family: Ericaceae

Botanical name: Vaccinium corymbosum
Common name: Blueberry

Parentage:

### FEMALE PARENT

Name: Draper (Duke unpatented×G751 unpatented)

U.S. Plant Pat. No. 15,103.

Compared to Draper, BB06-507MI-15 has a maturity date that is 3-5 days earlier. Also, Draper has a slightly darker colored berry that is larger and more flattened. The berry of BB06-507MI-15 is much firmer and sweeter flavored than Draper

### MALE PARENT

Brigitta (open pollinated selection of Lateblue unpatented)

U.S. Plant Pat. No.: none

Compared to Brigitta, BB06-507MI-15 matures 14 days earlier. The fruit of BB06-507MI-15 is slightly smaller and firmer and has a lighter blue color than Brigitta. The bush habit of BB06-507MI-15 is more upright.

BB06-507MI-15 was created from a cross in 2006 in a greenhouse in Holland, Mich. Emasculated flowers of Draper were hand pollinated with pollen from Brigitta. Seed was germinated, and plants were grown for 18 months and then planted in a seedling evaluation plot near South Haven, Mich. in 2008. The plant was evaluated for 3 years and first selected in 2010, based on excellent and desirable fruit and morphological characteristics—maturity date, bush habit and vigor, berry quality, flavor, and storage ability. The bush was dug from the field in October 2011 and transplanted into a pot and placed in a virus-free screened greenhouse. Additional plants have propagated by softwood cuttings and tissue culture and have retained the original characteristics. Five and ten bush advanced trials have been planted in Grand Junction, Gobles, and South Haven, Mich.

BB06-507MI-15 was first asexually propagated by softwood cuttings in 2011 in Grand Junction, Mich. Micropropagation by tissue culture was also first initiated in 2011 and produced cloned plants. The propagated plants from softwood cuttings and tissue culture have retained the original characteristics. The variety roots readily from softwood cuttings and tissue culture microshoots.

Field observations were made in 2012 on a mature 6-year old plant located in Grand Junction, Mich. Laboratory analysis of fruit characteristics were also done in Grand Junction.

General comments: BB065-507MI-15 is a new and distinct high chill tetraploid Northern highbush blueberry (Vaccinium) variety of complex ancestry, based largely on V. corymbosum with a limited number of genes from V. darrowii, V. angustifolium, V. tenellum, and V. ashei. It is a productive early ripening variety that ripens approximately at the same time as Duke, and 10 days before Bluecrop. The variety Duke in Michigan is troubled by winter hardiness and plant vigor issues. BB065-507MI-15 is intended to replace Duke in Michigan with much improved winter hardiness, plant vigor, and berry quality. BB065-507MI-15 is characterized as having large fruit of medium light blue color, very firm with a very small and dry picking scar. The fruit is well exposed on a vigorous upright plant. Along with a small crown, a loose berry cluster, concentrated ripening and easily detached fruit, the variety displays characteristics suitable for mechanical harvesting. It is intended for areas that successfully grow high chill Northern highbush varieties. Ripe fruit are large, typically 2.5 grams/berry. The mean date of flowering in southern Michigan is approximately May 1. Frost protection may be needed for successful pollination and fruit set. Winter chill requirement for successful flowering and leafing is at least 1000 hours below 7° C. The mean ripening date is July 1. Fruit shape is oblate with a medium high amount of waxy bloom that is rather persistent following handling. It has very good flavor, balanced between medium levels of sweetness and acidity with a crunchy and juicy texture. It has above average storage ability in refrigeration of 3-4 weeks. BB065-507MI-15 provides a very good yield potential, a very good plant vigor, fruit quality and firmness, as well as characteristics suitable to mechanical harvesting, and very good flavor and texture.

References to color refer to The Pantone Book of Color, Eisemann and Herbert, Harry N. Abrams, Inc. Publishers, New York, ISBN 0-8109-3711-5, 1990.

SpectraMagic NX Model CR410, Konica Minolta, Japan.

Morphological characteristics reference: Plant Systematics, Jones and Luchsinger, 2 Ed., McGraw Hill, New York, ISBN 0-07-032796-3, 1986.

Firmness readings—BioWorks FirmTec2, Wamena, Kans.
Average size information: Medium large bush, very upright shape, 6-year old plant 170 cm height, 49 cm width, height/width ratio 3.5:1.
Growth: Very good.
Productivity: Very good.
Cold hardiness: Leaf and flower buds −26° C., open flowers and fruit −2° C. Specific features of the variety:
Plant:
Growth habit: Very upright.
Plant width: 49 cm.
Plant height: 170 cm.
Spread: 65 cm.
Productivity: 5-6 lbs per mature bush.
Cold hardiness/tolerance: Leaf and flower buds −26° C., flowers and fruit −2° C.
Chilling requirement: 1000+ hours below 7° C.
Canes: Modestly branched, 7-8 canes/bush, 51-98 cm range, average 65 cm; medium number of laterals.
   Mature cane color.—Pantone Moon Beam 13-0000.
Texture: medium rough.
Fruiting wood: Smooth, immature winter color — Pantone Red Ochre 18-1442; immature summer color — Pantone Herbal Garden 15-0336.
Internode length 12-20 cm range, average 16 mm.
Surface texture of new wood: Smooth.
   Mature canes .—circular, 15 mm width.
Time of beginning of leaf bud burst (include location(s)): April 15 (Grand Junction, Mich.).
Time of beginning of flowering (include location(s)): April 25 (Grand Junction, Mich.).
Time of fruit ripening (include location(s)): July 1 (Grand Junction, Mich.).
Disease resistance/susceptibility: None claimed.
Foliage:
Leaf color: Upper — Pantone Black Forest 19-0315; lower — Pantone Peridot 17-0336.
Leaf arrangement: Alternate.
Leaf margins: Entire.
Leaf venation: Pinnate.
Leaf apices: Acute.
Leaf bases: Acute.
Vein and petiole colouration: Pantone Herbal Garden 15-0336.
Petiole length: 2 mm.
Leaf dimensions:
Overall shape: 65 mm-95 mm range, average 72 mm.
Width: 35 mm-50 mm range, average 40 mm.
Leaf margins: Entire, no visible pubescence or nectaries.
Leaf surface: No visible pubescence.
Flower:
Flower shape: Elongate urceolate.
Flower bud number: Medium high.
Flowers per cluster: 9-10.
Flower fragrance: Light floral.
Corolla color: Pantone Turtle Dove 12-5202.
Corolla length: 8-9 mm.
Corolla aperture width: 3 mm.
Flower peduncle: 16 mm.
Color: Pantone Leek Garden 15-0628.
Flower pedicel: 7 mm.
Color: Pantone Leek Garden 15-0628.
Calyx (with sepals): 2 mm.
Color: Pantone Leek Garden 15-0628.
   Stamen.13 Length: 7 mm.
Number per flower: 10.
Filament color: Pantone Raw Sienna 17-1436.
Style: 8 mm, top of ovary to stigma tip.
Color: Pantone Herbal Garden 15-0336.
Pistil: 7 mm.
Ovary color: Pantone Herbal Garden 15-0336.
   Anther.13 Length: 3 mm.
Number: 10.
Color: Pantone Raw Sienna 17-1436.
Pollen:
Abundance: medium high.
Color: Pantone Ivory 11-0907.
Fruit:
Date of 50% maturity: July 1 (Grand Junction, Mich.).
Yield: 5-6 lbs/bush.
Berry color.
With wax: Pantone Provincial Blue 18-4220.
With wax removed: Pantone Majolica Blue 19-4125.
Berry flesh color: Pantone Frozen Dew 13-0513.
Berry surface wax abundance: Medium-heavy, medium persistent.
Calyx: width — 5 mm; depth — 2 mm; shape — 5 lobed, ridging of 1.5 mm of lobes, turned inward.
Berry weight: 2.4 grams/berry.
Berry size diameter: 18 mm width, 12 mm height, Aspect (H/W) — 0.7.
Berry shape: Oblate.
   Cluster density.—loose.
   Detachment force. —medium easy.
Self-fruitfulness.—good; will require cross pollination for maximum, yield, and earliness.
Fruit stem scar: Small, 2 mm, dry.
Berry firmness: Very firm; FirmTec2 reading — 219 g/mm².
Berry sweetness: Medium, high Brix° 12.1.
Berry acidity: Medium, TA — 0.68.
Berry flavor and texture: Very good flavor with balanced sweetness and acidity, crunchy and juicy texture.
Suitability for mechanical harvesting: Very good due to fruit firmness, bush shape, crown size, berry detachment.
Seed:
Seed abundance in fruit: Medium.
Seed color: Pantone Butterscotch 15-1147.
Seed dry weight: NA.
Seed size: Medium 1 mm.
Possible typical Market uses: Fresh market, processing into jams, puree, yogurt.
Storage quality: Very good, 3-4 weeks in refrigerated storage.

What is claimed is:

1. A new and distinct cultivar of Blueberry plant named BB06-507MI-15 as described and shown herein.
